(19) United States
(12) Patent Application Publication
(10) Pub. No.: US 2018/0161388 A1
(43) Pub. Date: Jun. 14, 2018

(54) SUBSTANCE P, MAST CELL DEGRANULATION INHIBITORS, AND PERIPHERAL NEUROPATHY

(71) Applicant: Beth Israel Deaconess Medical Center, Inc., Boston, MA (US)

(72) Inventors: Lijun SUN, Harvard, MA (US); Aristidis VEVES, Quincy, MA (US)

(21) Appl. No.: 15/574,913

(22) PCT Filed: May 17, 2016
(86) PCT No.: PCT/US2016/032836

§ 371 Date: Nov. 17, 2017

Related U.S. Application Data
(60) Provisional application No. 62/162,972, filed on May 18, 2015.

(51) Int. Cl. A61K 38/04; A61P 25/02; A61P 17/02; A61K 9/00

(52) U.S. Cl. CPC A61K 38/046; A61K 9/0014; A61P 17/02; A61P 25/02

(57) ABSTRACT

Described herein are methods of using Substance P, mast cell degranulation inhibitors, or combinations thereof to delay the onset of, to reverse, or to reduce the risk of acquiring complications associated with diabetes. Also provided herein are methods for accelerating wound healing in diabetic subjects using Substance P, mast cell degranulation inhibitors, or combinations thereof.

### RELATED APPLICATIONS

This application claims the benefit of the filing date of U.S. Provisional Application No. 62/162,972, filed May 18, 2015, the entire contents of which are incorporated herein by reference.

### TECHNICAL FIELD OF THE INVENTION

Provided herein is the role of local skin inflammation on the development of small fiber neuropathy and methods of using Substance P (SP), mast cell (MC) degranulation inhibitors, or a combination thereof, to delay the onset of, to reverse, or to reduce the risk of acquiring complications associated with diabetes. Also provided are methods for accelerating wound healing in diabetic subjects using Substance P, mast cell degranulation inhibitors, or combinations thereof.

### BACKGROUND

Diabetic peripheral neuropathy (DPN) is the most common complication of diabetes, clinically affecting approximately 50% of patients. See e.g., Neurology 1995; 45:1115-21. It is the main etiopathogenic factor in serious conditions such as painful neuropathy and foot ulceration often followed by lower extremity amputation. See e.g., Pain medicine 2008; 9:660-74 and Diabetes Care 2000; 23:606-11. Despite extensive efforts employing aldose reductase inhibitors, antioxidants, nerve growth factors and protein kinase C β inhibitors, there is no FDA approved treatment to modify or reverse disease progression. Rather, the only proven technique that modifies the development and progression of diabetic neuropathy is good glycemic control. See e.g., Cochrane Database Syst Rev 2012; 6:CD007543 and J Peripher Nery Syst 2012; 17 Suppl 2:22-7.

Small fiber neuropathy (SFN) is part of DPN and affects the somatic thinly myelinated Aδ, unmyelinated C and autonomic nerve fibers (Current diabetes reports 2012; 12:384-92). It may be the first abnormality of nerve dysfunction in diabetes and it can be accurately assessed by evaluating the intraepidermal nerve fiber density (IENFD). See e.g., Journal of neurology 2008; 255:1197-202; Muscle Nerve 2007; 35:591-8; Journal of the neurological sciences 1993; 115:184-90; and Diabetes/metabolism research and reviews 2011; 27:678-84. Recent studies have shown that the deterioration in IENFD is not reversed in type 1 diabetic patients (T1DM) who undergo pancreas transplantation and achieve normoglycemia, indicating early intervention is required to prevent the development of SFN. See e.g., Diabetes Care 2008; 31:1611-2 and Diabetes 2009; 58:1634-40.

Over the last decade, it has become apparent that inflammation is a major factor of diabetic neuropathy (Nature reviews Neurology 2011; 7:573-83) Dyslipidemia (Diabetes 2009; 58:1634-40), LDL oxidation (Diabetes 2009; 58:2376-85), poly(ADP-ribose) activation (Free Radic Biol Med 2011; 50:1400-9) and increased levels of advanced glycated endproducts (AGEs) and their receptor RAGE (Diabetes 2013; 62:931-43) are the main causes for this increased inflammatory response (Diabetologia 2009; 52:2251-63). Also, reduction of inflammation in animal models of diabetic neuropathy using various factors such as neutralization of TNF-α increased the IENFD. See e.g., American journal of physiology Endocrinology and metabolism 2011; 301:E844-52. Taken together, these data have provided proof of concept that reducing inflammation may be a reasonable new therapeutic approach.

However, almost all interventions to treat diabetic neuropathy have been based on the systemic administration of tested agents in both human (Diabetes Care 2009; 32:1256-60, Diabetes Care 2011; 34:2054-60, and JAMA 2000; 284:2215-21) and experimental diabetes (Diabetologia 2006; 49:3085-93, General physiology and biophysics 2010; 29:50-8, Brain Res 1994; 634:7-12, and Diabetologia 2010; 53:1506-16). Given the problems associated with systemic treatments, and the serious morbidity and mortality rates associated with SFN, the need to identify new factors in the development of SFN and therapeutic treatments to target such factors, and ultimately to reverse to progression of SFN non-systemically, remains.

Here, the subject application focuses in part, on the role of local skin inflammation on the development of SFN, and identifies several new factors that play a role in development of SFN and DPN, such as e.g., the interaction among neuropeptides, mast cells and macrophages, and in particular increased mast cell degranulation and M1 macrophage activation in diabetic models. See e.g., FIG. 16.

### SUMMARY

It has now been found that the interaction among neuropeptides, mast cells and macrophages, and events such as increased mast cell degranulation and M1 macrophage activation, play a significant role in diabetic peripheral neuropathy models. See e.g., FIGS. 1-3. In one aspect, mast cell degranulation is increased in diabetic patients while Substance P production is reduced.

Without wishing to be bound by theory, mast cell degranulation in diabetic patients (in particular the increase thereof) is a main factor associated with skin inflammation and related conditions, and systemic and/or topical MC stabilization prevents or reverses complications associated with diabetes (such as e.g., diabetic small fiber neuropathy) or heals wounds in diabetic patients (such as e.g., foot ulcers).

The use of mast cell degranulation inhibitors and/or Substance P for reversing the progression of small fiber neuropathy (SFN) has now been found. Such methods include e.g., topical and/or non-systemic administration of a mast cell degranulation inhibitor, Substance P, or a combination thereof for modulating (MC) degranulation and M1 macrophage activation. See e.g, FIG. 14.

In one aspect, provided herein are methods of delaying the onset of, reversing, or reducing the risk of acquiring peripheral neuropathy (PN) in a subject (such as in a human having diabetes), comprising administering to the subject a therapeutically effective amount of a Substance P, a mast cell (MC) degranulation inhibitor, or a combination thereof.

Further aspects relate to accelerating the healing of a wound (such as a foot ulcer) in subjects (such as in a human having diabetes), comprising administering to the subject a therapeutically effective amount of Substance P, a mast cell (MC) degranulation inhibitor, or a combination thereof

### BRIEF DESCRIPTION OF THE FIGURES

FIG. 1 illustrates that Substance P (SP) is reduced in diabetic patients and mice.

FIG. 2 illustrates the number of degranulated skin mast cells is increased in diabetic patients and is associated with inflammation where a) represents non-degranulated (Non-DM) and degranulated (DM) mast cells (MC) in forearm human skin biopsies where degranulated cells were in proximity with inflammatory cells that were increased in DM patients; b) represents the total MC count was increased in the diabetic patients (DM) when compared to the healthy control subjects (non-DM) (*p<0.05); c) shows the number of degranulated MC was also increased in DM (**p<0.01); d) shows the number of non-degranulated MC was reduced in the DM (*p<0.05); e) shows dermis inflammatory cells as a function of degranulated MC; f) shows IL-6 as a function of degranulated MC; and g) shows TNFα as a function of degranulated MC.

FIG. 3 illustrates an increased M1/M2 ratio at the foot skin of DM patients

FIG. 4 illustrates an increase in the expression of the M1-associated pro-inflammatory cytokines in the foot skin of diabetic patients where a) represents TNF-alpha; (b) represents IL-1beta; and c) represents that gene expression of the M2-associated anti-inflammatory cytokine IL-10 was reduced in the foot skin of diabetic patients.

FIG. 5 illustrates differences in IENFD levels in healthy and diabetic neuropathy patients where C) is normal IENFD in a healthy individual and where DM-PDN shows reduced IENFD in an patient with diabetic neuropathy.

FIG. 6 illustrates IENFD was reduced at the distal leg to pathological levels in many individuals with type 1 diabetes mellitus, and most with type 2 diabetes mellitus.

FIG. 7 illustrates SP gene expression was reduced and neutral endopeptidase (NEP) enzyme increased in streptozotocin (STZ) induced diabetic mellitus mice.

FIG. 8 illustrates the number of degranulated MC are increased in the skin of STZ induced diabetic mellitus mice where a) represents non-degranulated (black arrows) and degranulated (red arrows) mast cells (MC) from non-diabetic mellitus (non-DM) and STZ induced diabetic mellitus mice (STZ-DM), treated or non-treated with the MC stabilizer disodium cromoglycate (DSCG); b) represents extensively degranulated MC; and c) represents non-degranulated MC.

FIG. 9 illustrates staining for M1 and M2 macrophages in mice skin where a) represents that M1/M2 macrophage ratio was increased in STZ induced diabetic mellitus mice; and where b) and c) represents non-diabetic and diabetic NK1RKO and TAC1KO mice.

FIG. 10 illustrates that treatment with DSCG has no effect on the M1/M2 ratio of non-DM mice but drastically reduces it in STZ-DM to normal levels.

FIG. 11 illustrate that IL-6 skin gene expression was increased in STZ-DM, non-DM, STZ-DM NK1RKO and TAC1KO mice as shown by a) and b). Similar results were observed in the KC (equivalent to human IL-8) gene expression as shown by c) and d).

FIG. 12 illustrates topical SP application (red color) in wounds of non-DM (left panel) and diabetes mellitus (DM) mice (right panel) induced an acute inflammatory response at Day-3 (as seen by the IL-6 and M1/M2 ratio response) and reduced the chronic inflammation in the DM mice at Day-10.

FIG. 13 illustrates IENFD using PGP9.5 staining in a diabetic mice (DM) and a diabetic mice treated with topical SP application (DM-SP) for 10 days.

FIG. 14 illustrates topical SP application in normal skin in periwound area of non-DM mice (n=3) did not affect IENFD compared to non-treated, non-DM (n=4). DM mice (n=4) non-treated mice tended to have lower IENFD that returned to normal levels in SP-treated DM mice which was non-significant due to small number of animals.

FIG. 15 illustrates that neuropathic groups had higher serum levels leptin, G-CSF (p<0.05), sE-Selectin, sICAM, sVCAM, CRP, TNFα and fibrinogen.

FIG. 16 illustrates certain events associated with diabetes.

### DETAILED DESCRIPTION

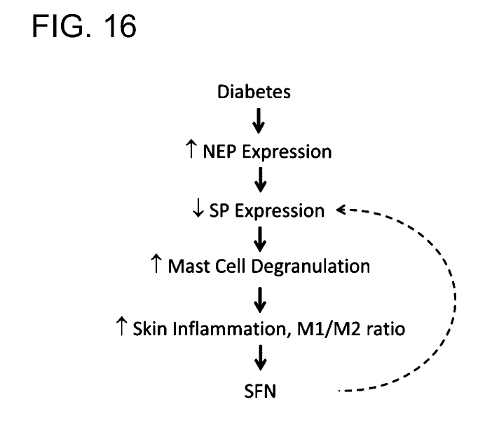

Without being bound by theory, the present disclosure relates to the finding that reduced Substance P (SP) skin expression in diabetic patients leads to a chronic local inflammatory state and mast cell degranulation and macrophage activation, which in turn causes small fiber neuropathy (SFN). A general schematic of this finding with additional elements is provided by FIG. 16.

Also provided herein, without being bound by theory, is the finding that local application of Substance P and/or mast cell degranulation inhibitors can prevent or reverse small fiber neuropathy.

Thus, in one aspect, the present disclosure provides a method of delaying the onset of, reversing, or reducing the risk of acquiring peripheral neuropathy (PN) in a subject having diabetes, comprising administering to the subject a therapeutically effective amount of Substance P, a mast cell (MC) degranulation inhibitor, or a combination thereof.

In another aspect, the present disclosure provides a method of delaying the onset of, reversing, or reducing the risk of acquiring peripheral diabetic neuropathy in a subject in need thereof, comprising administering to the subject a therapeutically effective amount of Substance P, a mast cell (MC) degranulation inhibitor, or a combination thereof.

In one aspect, the peripheral neuropathy in the methods described herein is small fiber neuropathy (SFN).

In one aspect, the subject of the methods described herein has Type 1 or Type 2 diabetes. In another aspect, the subject of the methods described herein has diabetes mellitus type 2.

In one aspect, the mast cell degranulation inhibitor, Substance P, or combination thereof of the methods described herein is administered topically.

In one aspect, the mast cell degranulation inhibitor of the methods described herein is a calcium channel blocker or receptor potential canonical (TRPC) channel blocker. In another aspect, the mast cell degranulation inhibitor of the methods described herein is a calcium-release activated calcium (CRAC) channel blocker.

The determination, diagnosis, and/or evaluation of peripheral neuropathy can be made according to standard guidelines, such as e.g., those defined in Diabetes Care 2010; 33:2285-93, Diabetes Care 2010; 33:2629-34, J Peripher Nery Syst 2013; 18:153-61, and The Journal of clinical endocrinology and metabolism 2009; 94:2157-63. In particular, this may include e.g., symptom evaluation using the Neuropathy Symptom Score (NSS) and Utah Early Neuropathy Scale questionnaires (J Peripher Nery Syst 2008; 13:218-27), physical examinations quantified by the Neuropathy Disability Score (NDS) and NIS (LL), Quantitative Sensory Testing using a MEDOC TSAII thermal and vibratory analyzer (Medoc Ltd., Israel), Nerve Conduction Studies using a Viking IIIP EMG instrument (Viasys Healthcare, Madison, Wis.), Autonomic Testing (Handbook of clinical neurology 2013; 115:115-36) and the nerve axon reflex-related vasodilation (NARV) by employing iontophoresis of 1% acetylcholine chloride with a DRT4 Laser-Doppler Blood Flow Monitor (Moor Instruments, Millwey, Devon, England). See e.g., Neurology 2003; 60:297-300 and Journal of neurology, neurosurgery, and psychiatry 2006; 77:927-32. Diabetic patients can be classified according to Toronto Criteria as described in e.g., Diabetes Care 2010; 33:2285-93. To ensure a group of subjects with a broad range of neuropathy severity, patient population will be classified into mild, moderate and severe neuropathy and approximately an equal number of patients from each category will be enrolled.

Examples of mast cell degranulation inhibitor include, but are not limited to, Cromoglicic acid, Beta2-adrenergic agonists (such as e.g., salbutamol, levosalbutamol, terbutaline, pirbuterol, procaterol, clenbuterol, metaproterenol, fenoterol, bitolterol mesylate, ritodrine, isoprenaline, salmeterol, formoterol, bambuterol, olodaterol, and indacaterol), ketotifen and salts thereof (such as e.g., ketotifen fumarate), methylxanthines, Pemirolast, Quercetin, Omalizumab, cromolyn sodium, Gastrocrom, Ketotifen Systemic, and Zaditen.

Examples of calcium channel blockers include, but are not limited to, dihydropyridines (e.g., Amlodipine, Aranidipine, Azelnidipine, Barnidipine, Benidipine, Cilnidipine, Clevidipine, Isradipine, Efonidipine, Felodipine, Lacidipine, Lercanidipine, Manidipine, Nicardipine, Nifedipine, Nilvadipine, Nimodipine, Nisoldipine, Nitrendipine, and Pranidipine), non-dihydropyridines (e.g., Verapamil, Gallopamil, and Fendiline), Benzothiazepine (e.g., Diltiazem), mibefradil, bepridil, flunarizine, fluspirilene, fendiline, gabapentin pregabalin, and ziconotide.

Examples of calcium-release activated calcium channel blockers include, but are not limited to, those described in e.g., WO 2005/009954 (e.g, Synta-66 (N-(2′,5′-dimethoxy-[1,1′-biphenyl]-4-yl)-3-fluoroisonicotinamide), WO 2010/122089 (e.g., (2,6-difluoro-N-(1-(2-phenoxybenzyl)-1H-pyrazol-3-yl)benzamide) and (2,6-difluoro-N-(1-(4-hydroxy-2-(trifluoromethyl)benzyl)-1H-pyrazol-3-yl)benzamide), U.S. Pat. No. 8,524,763, WO 2013/164769, WO 2013/164773, WO 2009/017819, WO 2011/042797, U.S. Pat. No. 8,377,970, U.S. Pat. No. 8,921,364, U.S. Pat. No. 8,623,871, U.S. Pat. No. 8,614,321, U.S. Pat. No. 7,816,535, WO 2012056478, WO 2011063277, and WO 2011042797.

Examples of receptor potential canonical (TRPC) channel blockers include, but are not limited to, SKF96365 and those described in e.g., WO 2006/023881, WO 2008/138126, U.S. Pat. No. 8,133,998, and US 2012/0264804.

As used herein, delaying the onset of, reversing, or reducing the risk of acquiring a condition recited herein (peripheral neuropathy (PN), small fiber neuropathy (SFN), and peripheral diabetic neuropathy) means decreasing the amount of mast cell degranulation in subjects who have elevated mast cell degranulation levels due to a condition/disease, such as e.g., diabetes. It has been found that subject having diabetes have an increase in mast cell degranulation.

As used herein, accelerating the healing of wound means that the mast cell degranulation inhibitor, Substance P, or combination thereof as described herein elicits a cellular environment that accelerates or promotes healing of the wound. For example, the mast cell degranulation inhibitor, Substance P, or combination thereof as described herein may elicit the release of cytokines such as CXCL8, CCL2 and CXCL7, each of which are necessary for the first phase of wound healing, thereby promoting healing of a wound. The first phase of wound healing is the inflammatory phase that lasts for approximately three days and it is followed by the proliferative phase that lasts two to three weeks. In chronic wounds this linear progression is abolished and are characterized by the presence of low grade chronic inflammation. The application of mast cell degranulation inhibitor, Substance P, or combination thereof can convert the chronic low grade inflammation to an intense acute inflammatory phase that then progresses to the proliferative phase and promotes wound healing.

### I. Measures of Small Fiber Function, Cutaneous Substance P Expression and Chronic Inflammation.

Systemic Inflammation is Associated with Peripheral Neuropathy and is More Pronounced in Painful Neuropathy that is Mainly Characterized by Small Fiber Neuropathy.

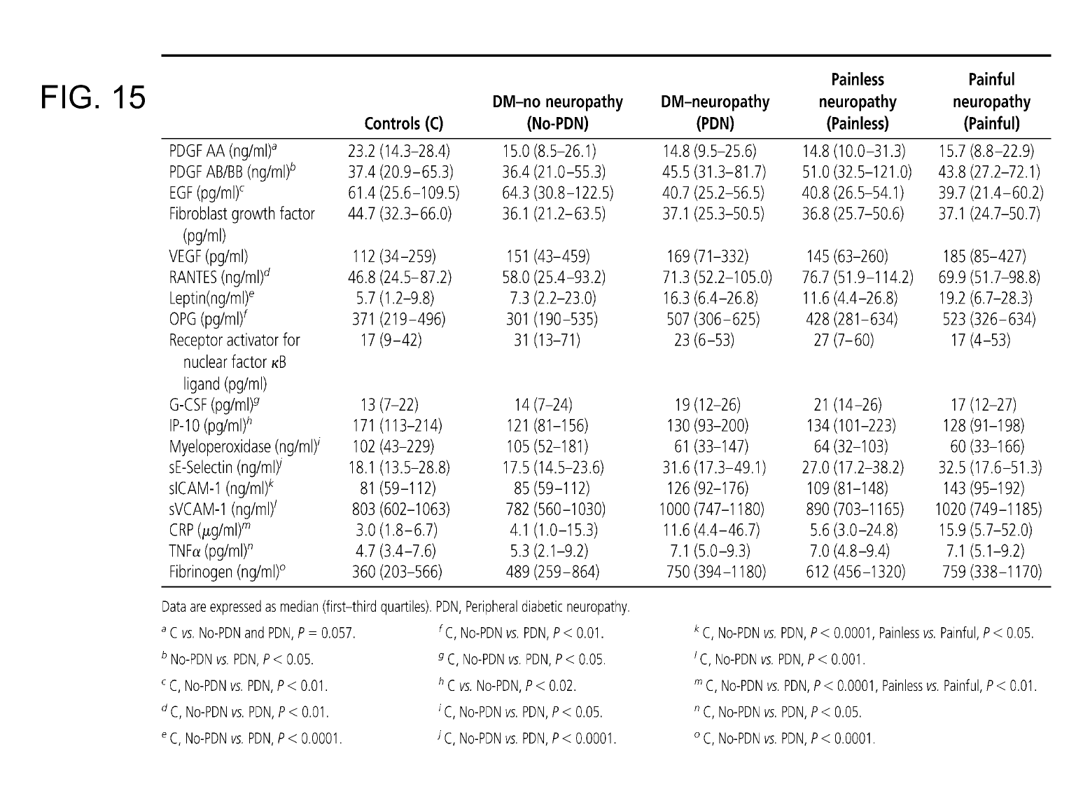

The following three groups were studied: 55 healthy control subjects, 80 non-neuropathic and 77 neuropathic DM patients. Neuropathic patients were subdivided to a subgroup of 31 subjects with painless neuropathy and 46 with painful neuropathy. As shown in FIG. 15, compared to the other two groups, the neuropathic group had higher serum levels leptin, G-CSF (p<0.05), sE-Selectin, sICAM, sVCAM, CRP, TNFα and fibrinogen. Patients with painful neuropathy had higher sICAM-1 (p<0.05) and CRP levels (p<0.01) when compared to painless neuropathy. These data have already been published. See also the Journal of clinical endocrinology and metabolism 2009; 94:2157-63.

Serum Substance P is Reduced in Diabetic Neuropathic Patients

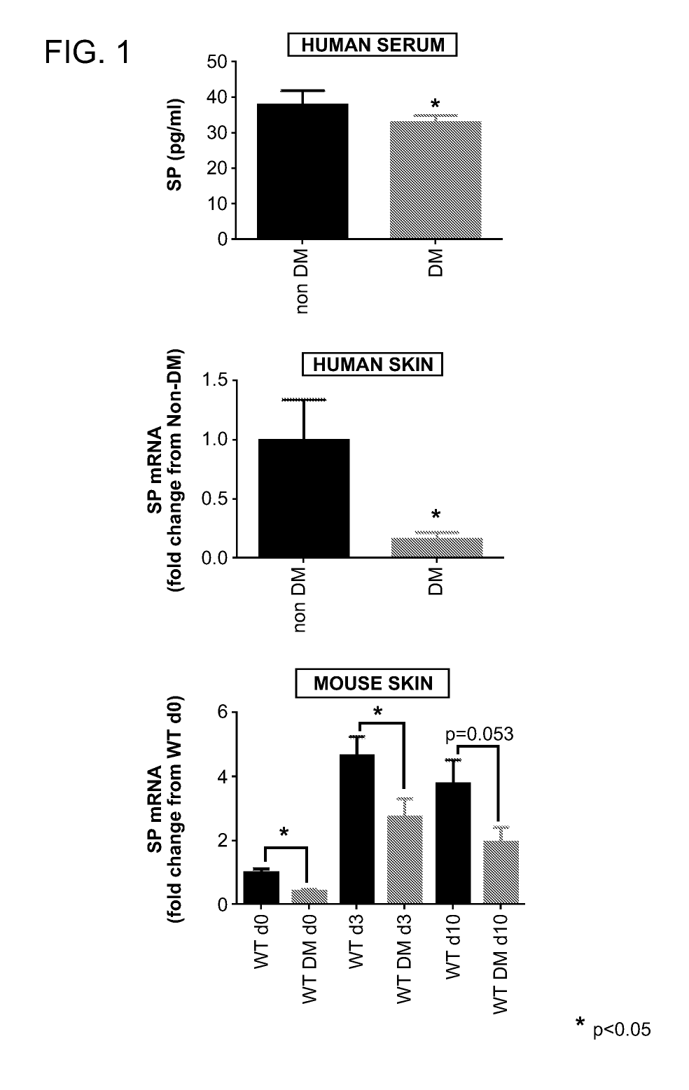

As shown by FIG. 1, serum SP is reduced in diabetic patients and mice.

Mast Cell (MC) Degranulation is Increased in Diabetic Patients and Correlates with Inflammation.

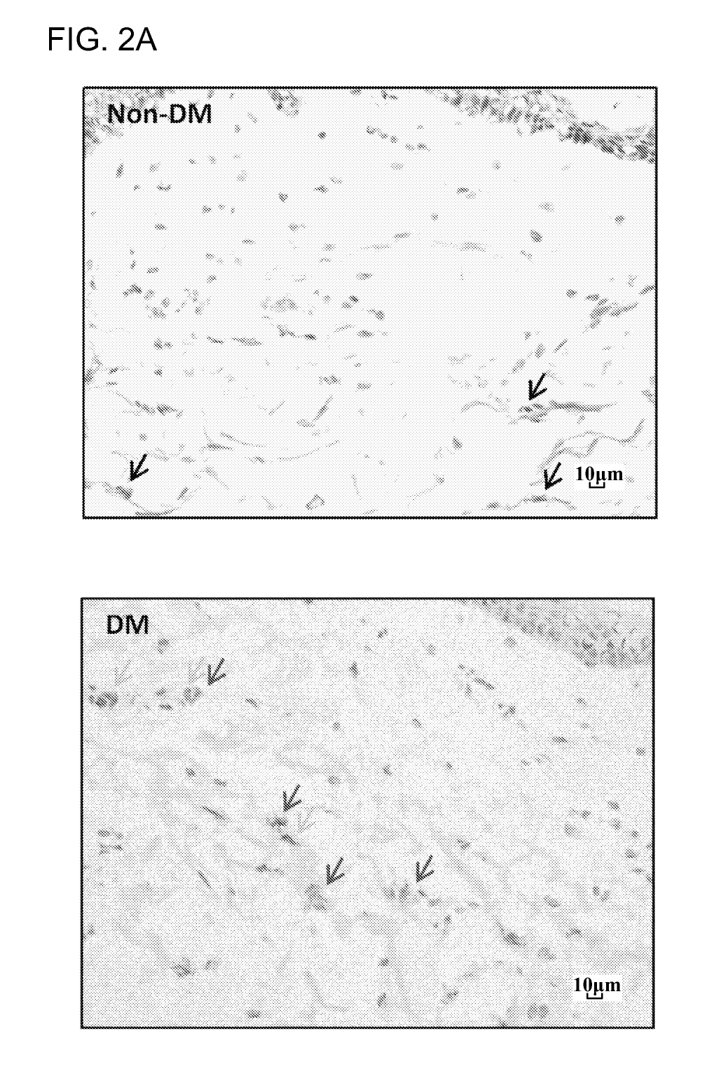

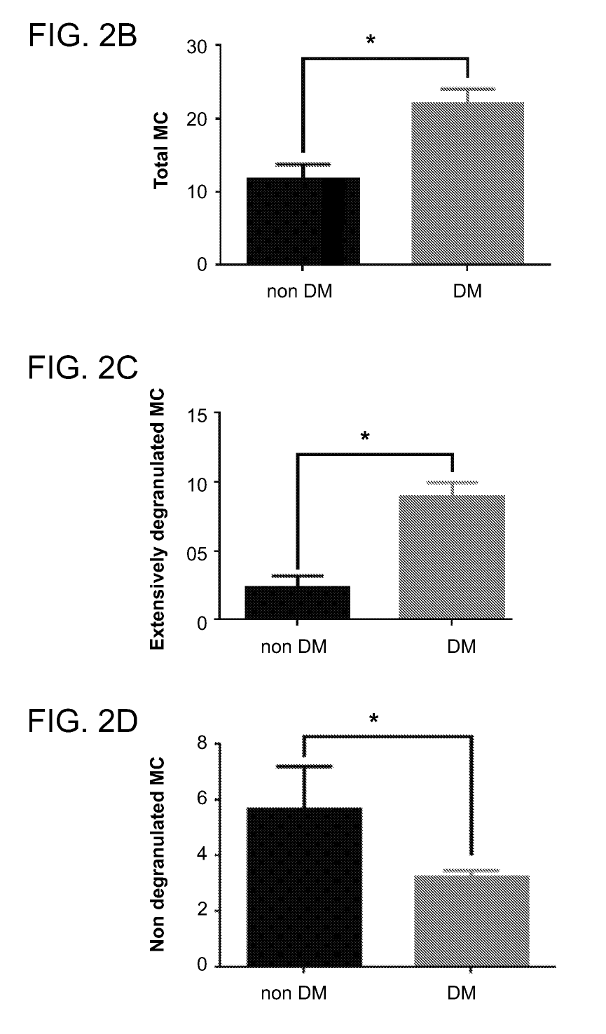

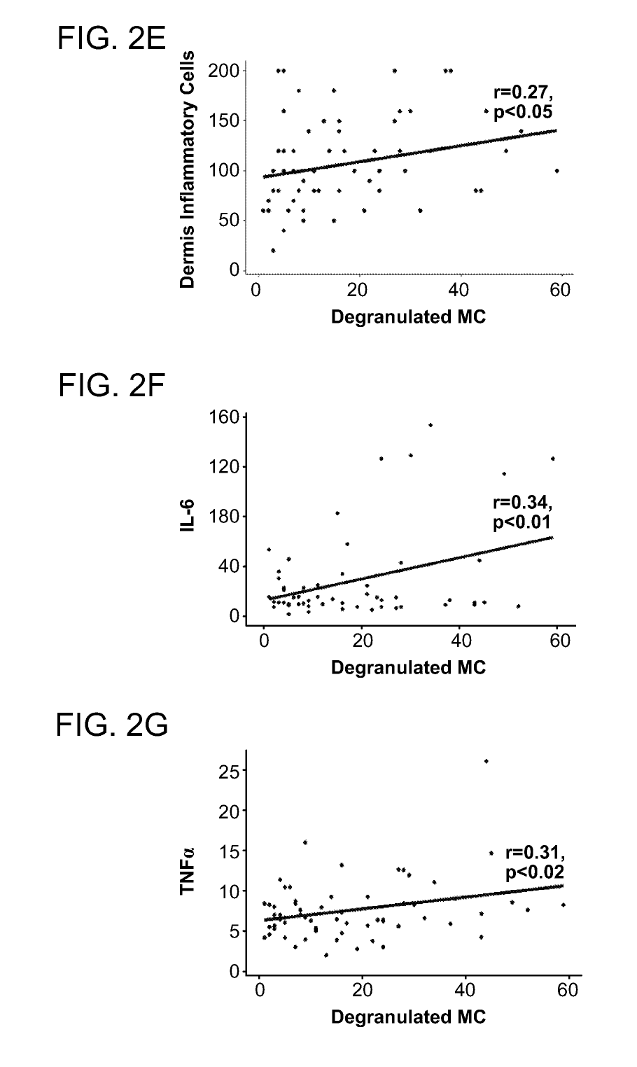

Non-granulated (black arrows) and degranulated MC (red arrows) in forearm skin were analyzed from skin biopsies from 10 healthy controls (non-DM) and 58 DM patients (FIG. 2) following the procedures described below and as previously defined. See e.g., Annals of neurology 2010; 67:534-41 and Annals of neurology 2010; 68:888-98. MC were stained with 0.1% Toluidine Blue using standard techniques (J Peripher Nery Syst 2008; 13:218-27, Handbook of clinical neurology 2013; 115:115-36, Immunol Rev 2007; 217:65-78, Proc Natl Acad Sci USA 2006; 103:7759-64, and Nature 1982; 297:229-31) and were found to be more degranulated compared to the control subjects. Of interest, Type 1 diabetes mellitus (T1DM) patients (n=25) had lower numbers of total and degranulated MC than Type 1 diabetes mellitus (T2DM) patients (19±11 vs 26±18, p=0.076) and (16±11 vs 22±17, p=0.095) respectively.

Macrophage Activation is Polarized Towards M1 in the Skin of Diabetic Patients.

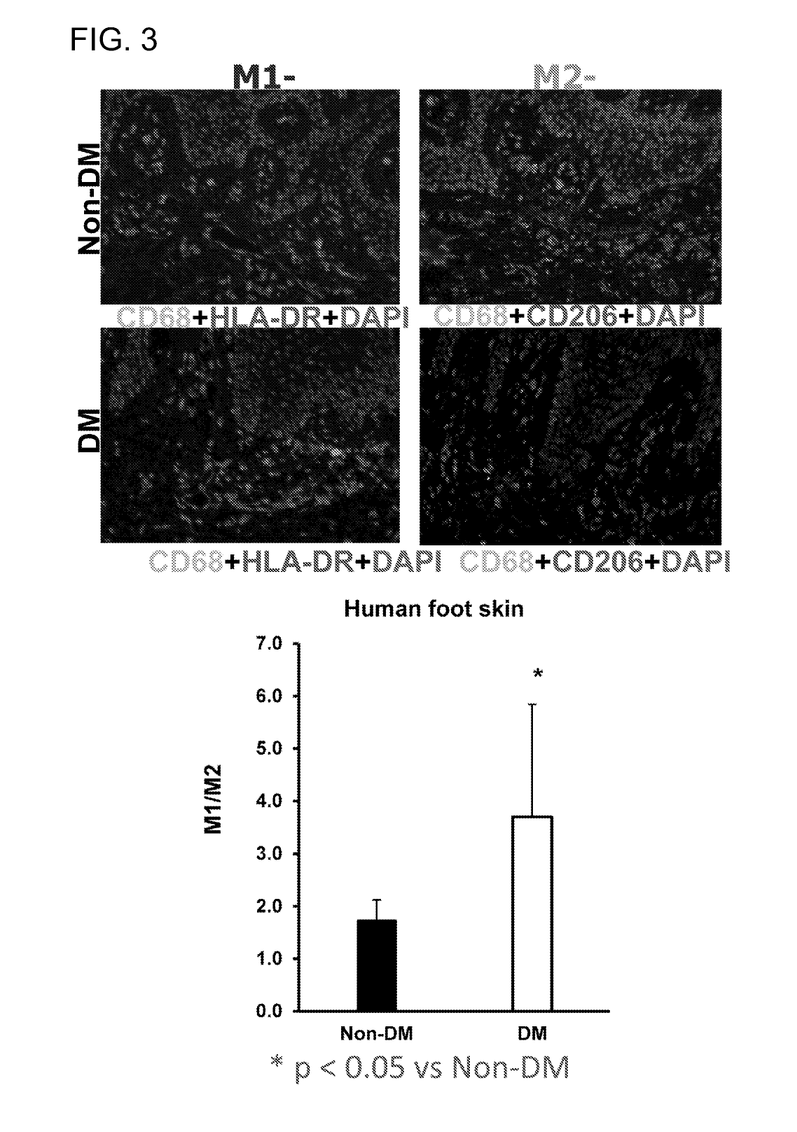

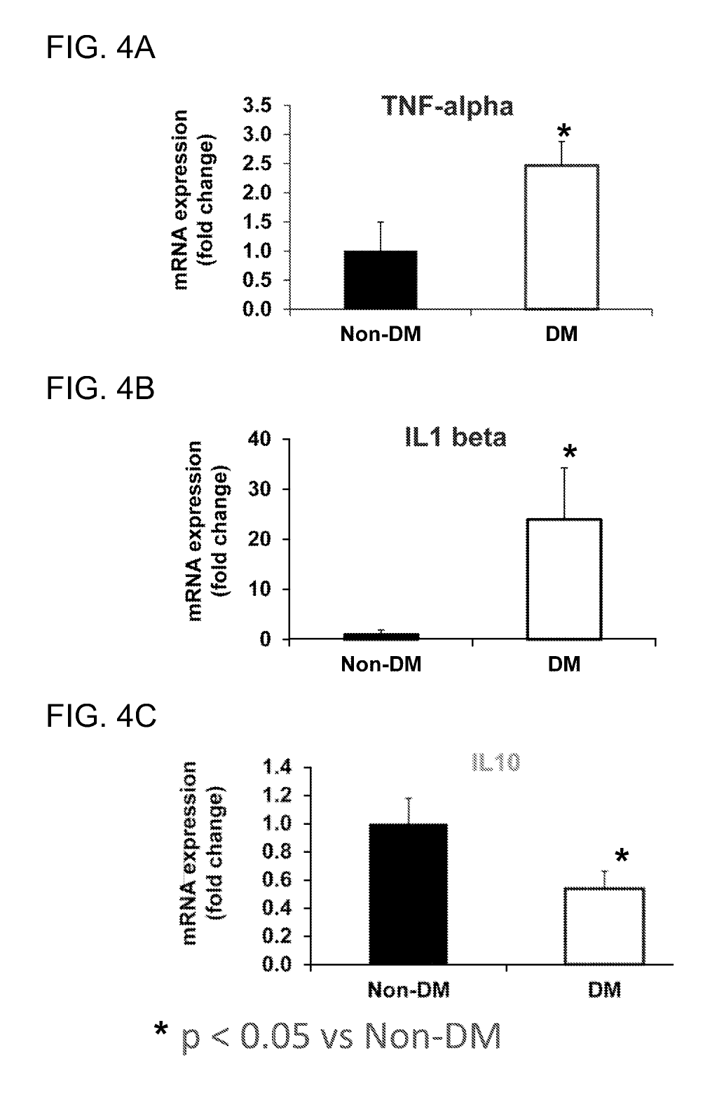

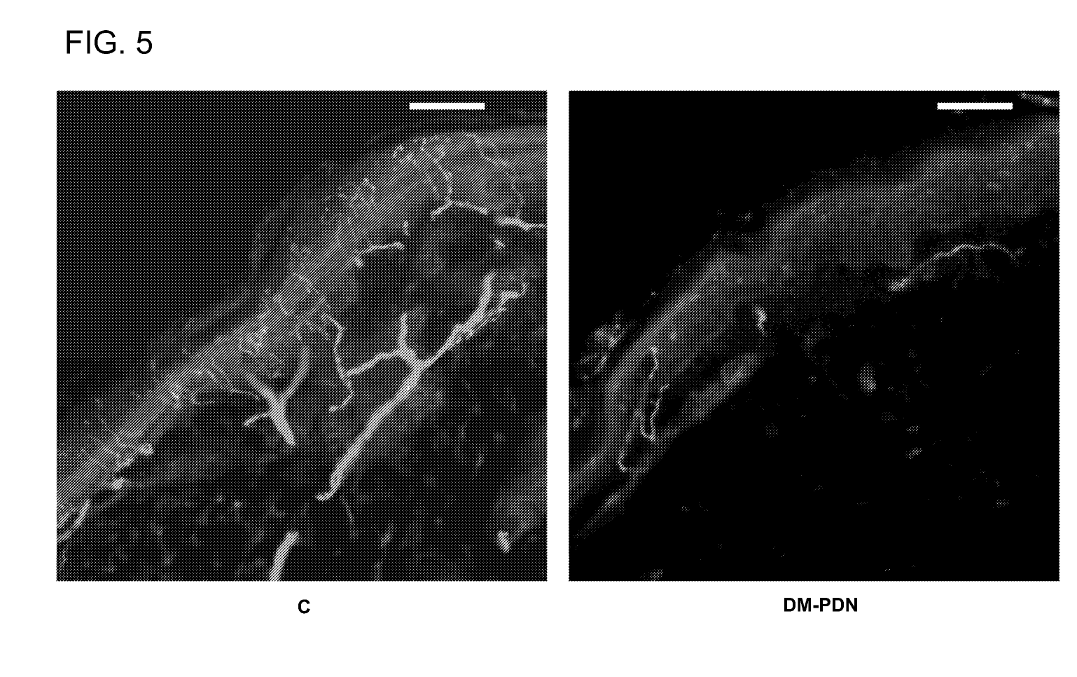

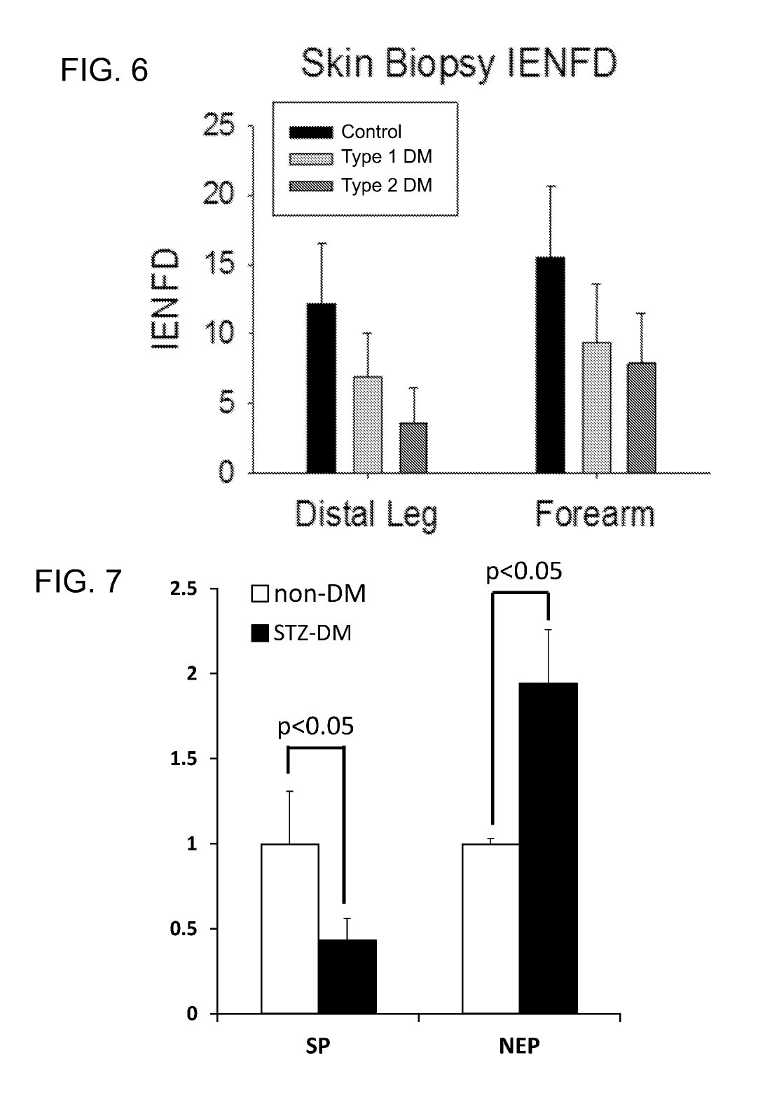

The number of HLA-DR⁺/CD68⁺ (M1−) and CD206/CD68⁺ (M2−) macrophages in the skin of DM patients and non-DM subjects were evaluated by immunofluorescence following previously defined methods. See e.g., J Peripher Nery Syst 2008; 13:218-27, Handbook of clinical neurology 2013; 115:115-36, Immunol Rev 2007; 217:65-78, Proc Natl Acad Sci USA 2006; 103:7759-64, and Nature 1982; 297:229-31. As shown in FIG. 3, there was an increased M1/M2 ratio at the foot skin of diabetes mellitus patients. The gene expression of M1-associated pro-inflammatory cytokines, such as TNF-α (FIG. 4, panel a) and IL-1β (FIG. 4, panel b), was also elevated in the foot skin of diabetes mellitus patients, whereas the M2-associated anti-inflammatory cytokine IL-10 was reduced (FIG. 4, panel c).

Intraepidermal Nerve Fiber Density is Reduced in Type 1 and Type 2 Diabetes Mellitus Patients.

Based on the above, and without wishing to be bound by theory, small fiber function appeared to be related to Substance P expressions, and inversely related to other markers of inflammation such as e.g., neutral endopeptidase enzyme expression, mast cell degranulation, and M1/M2 ratios. This is shown, in particular, by the data presented above where in diabetic patients, Substance P was reduced, neutral endopeptidase enzyme expression was increased, and mast cell degranulation was increased, which correlated to systemic inflammation and increased M1/M2 macrophage ratio at both the upper and lower extremities. As discussed above, inflammation is associated with diabetic neuropathy. See e.g., The Journal of clinical endocrinology and metabolism 2009; 94:2157-63, Diabetes Care 2009; 32:680-2, Diabetes Care 2013; 36:3663-70, Nature reviews Neurology 2011; 7:573-83, Diabetes 2009; 58:1634-40, and Diabetes 2009; 58:2376-85. Based in part on this new finding that mast cell degranulation is increased in diabetic patients, and given the correlation between mast cell degranulation, inflammation, and diabetic neuropathy, it was postulated (and indeed shown below) that the use of therapeutic stabilizers of mast cells in diabetic subjects would delay the onset of, reverse, or reduce the risk of the subject acquiring peripheral neuropathy.

### II. Efficacy of Local Small Fiber Neuropathy Treatment and Mast Cell Stabilization in Preventing or Reversing Small Fiber Neuropathy

a. Similar Correlations were Observed in STZ-DM Mice Models

C57BL/6J STZ-DM Mice have Reduced Skin SP and Increased NEP Expression.

Mice were diabetic for eight weeks. SP gene expression was reduced and NEP was increased in the DM mice when compared to their non-DM littermates (FIG. 7).

The Number of Degranulated MC is Increased in the Skin of STZ-DM Mice.

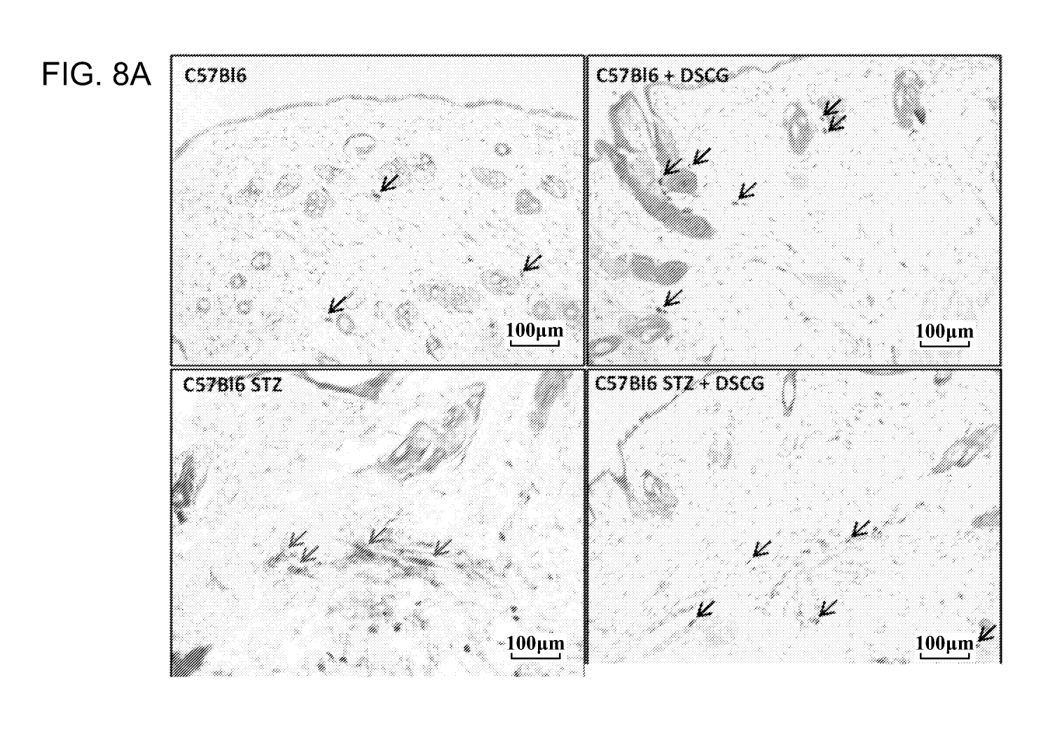

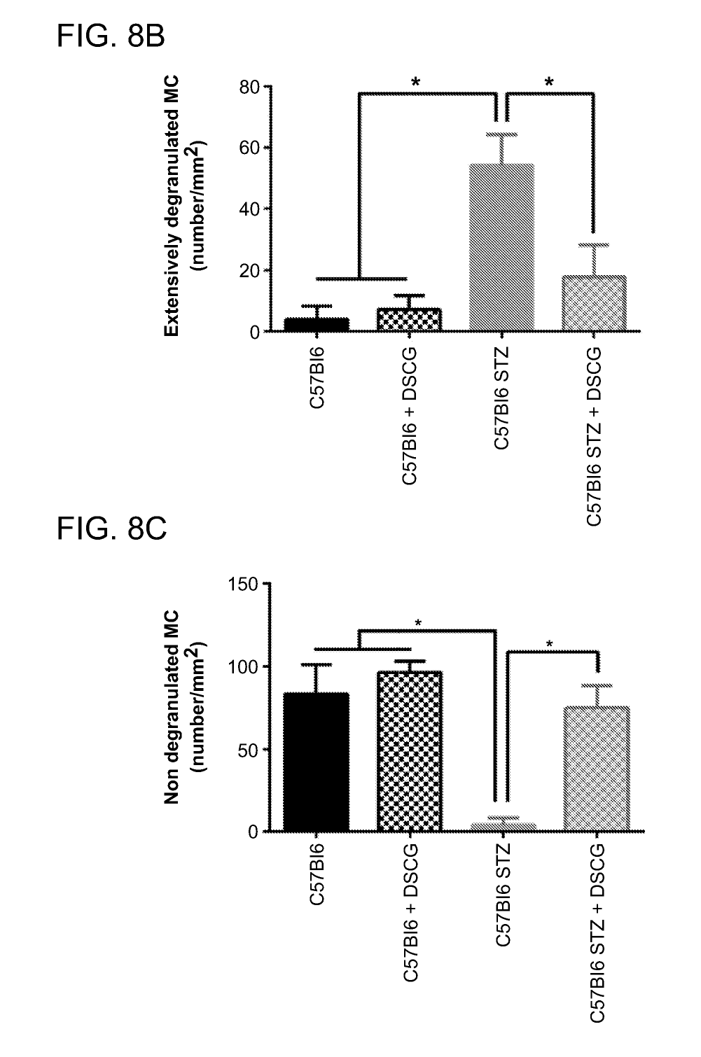

The number of intact or non-degranulated and degranulated MC in dorsal skin biopsies from non-DM and STZ-DM C57B16 mice with 8-weeks DM were evaluated by toluidine blue staining. Sections were stained metachromatically with 0.1% toluidine blue, pH 2 (cytoplasmic granules appear purple on a blue background). The cell number and extent of degranulation of mast cells was determined by a blinded observer and degranulation was scored as extensive (>50% of granules exhibiting fusion, alterations in staining, and extrusion from cell), moderate (10-50% of granules altered) or absent. See e.g., J Peripher Nery Syst 2008; 13:218-27, Handbook of clinical neurology 2013; 115:115-36, Immunol Rev 2007; 217:65-78, Proc Natl Acad Sci USA 2006; 103:7759-64, and Nature 1982; 297:229-31. Non-DM and STZ-DM mice were also treated for ten days intraperitoneally with the MC stabilizer disodium cromoglycate (DSCG). No differences were observed in the total counts of MC between non-DM and STZ-DM. However, the number of extensively degranulated MC was increased whereas the number of non-degranulated cells was reduced in STZ-DM mice compared to the non-DM controls. Disodium cromoglycate was able to effectively reduce the number of degranulated cells in STZ-DM mice (FIG. 8).

The M1/M2 Macrophage Ratio is Increased in STZ-DM Mice and KO Mice not Expressing SP (TAC1KO) and its NK1 Receptor (NK1RKO).

Using the same methods as in the human samples described above, an increased M1/M2 ratio in C57BL/6J STZ-DM mice was observed. In order to evaluate the role of SP in these changes, tachykinin precursor 1 deficient mice (Tac1^{-/-}) that do not express SP and mice deficient in the main receptor through which SP exerts its action, the receptor NK-1R (NK1R^{-/-}), were also tested. Both non-DM and STZ-DM KO mice and their normal littermates were tested.

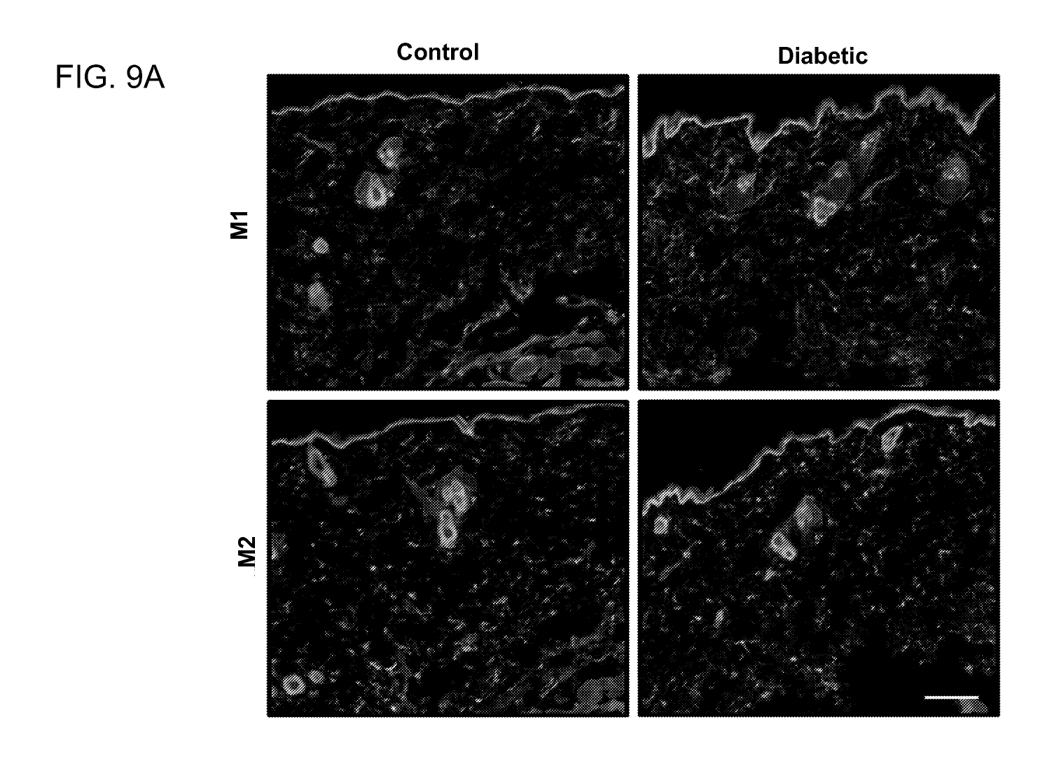

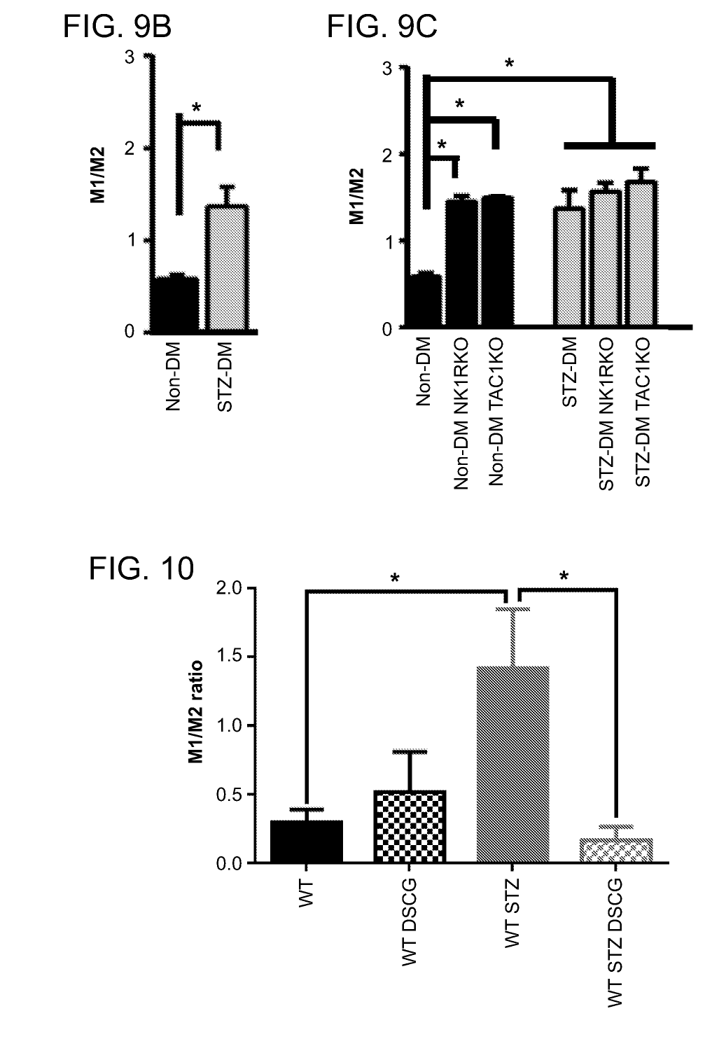

C57BL/6J STZ-DM mice had a higher M1/M2 ratio when compared to non-DM (FIG. 9). In addition, when compared to their littermates, non-DM NK1RKO and TAC1KO had increased M1/M2 ratio. In agreement with the results observed in the C57BL/6J mice, the STZ-DM littermates had also increased M1/M2 ratio when compared to non-DM littermates. No differences existed between the STZ-DM normal littermates and the KO mice.

b. MC Stabilization

MC Stabilization with Disodium Cromoglycate Reduces the M1/M2 Ratio.

The effect of a 10-day disodium cromoglycate (DSCG) intraperitoneal administration was evaluated in non-DM and STZ-DM C57B16 mice that were also tested for their wound healing capacity. Measurements performed ten days after treatment completion showed that DSCG treatment had no effect on non-DM mice (FIG. 10). See also Wound Repair and Regeneration 2013; 21:A45-A. However, STZ-DM mice not treated with DSCG had a high M1/M2 ratio while DSCG treatment reduced the ratio to levels similar to the non-DM mice.

Diabetes and Deficiency of SP or its Receptor NK1R Increase Skin Inflammatory Cytokine Gene Expression.

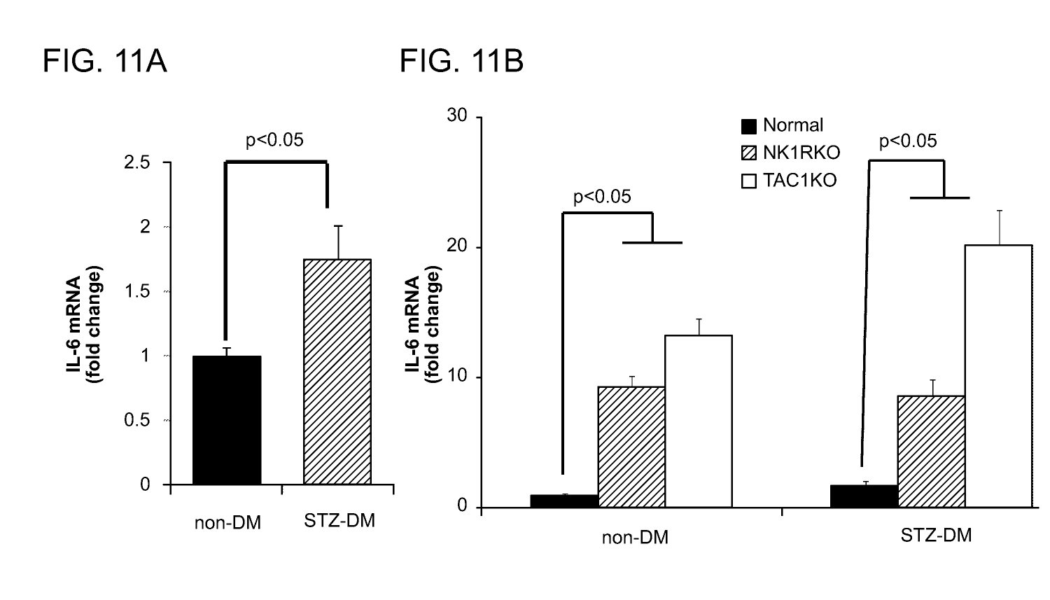

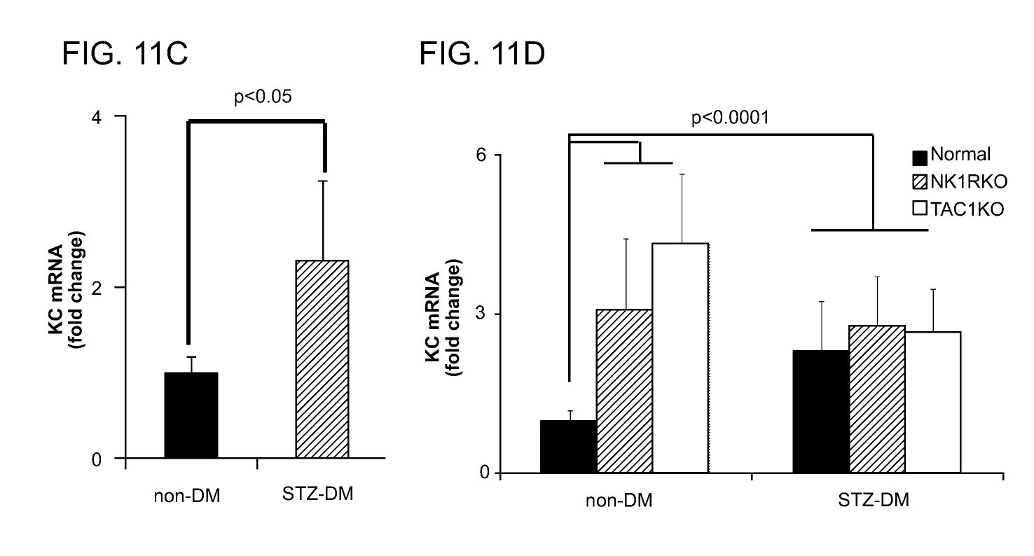

The IL-6 and KC (equivalent to human IL-8) skin gene expression was evaluated and it was found that they were both increased in STZ-DM C57B16 and in both non-DM and STZ-DM NK1RKO and TAC1KO mice (FIG. 11). Of interest, diabetes did not result in any changes in the KO mice. This result supports that diabetes induces chronic inflammation at the skin level. Furthermore, deficiency of SP or its receptor NK1R induces an even stronger inflammation that is not further affected by the induction of diabetes. This supports that the diabetes-induced SP deficiency is a major factor for the observed results.

Local Application of Substance P Improves Wound Healing by Inducing an Acute Early Inflammatory Response and Reducing Diabetes-Related Chronic Inflammation in Later Wound Healing Stages.

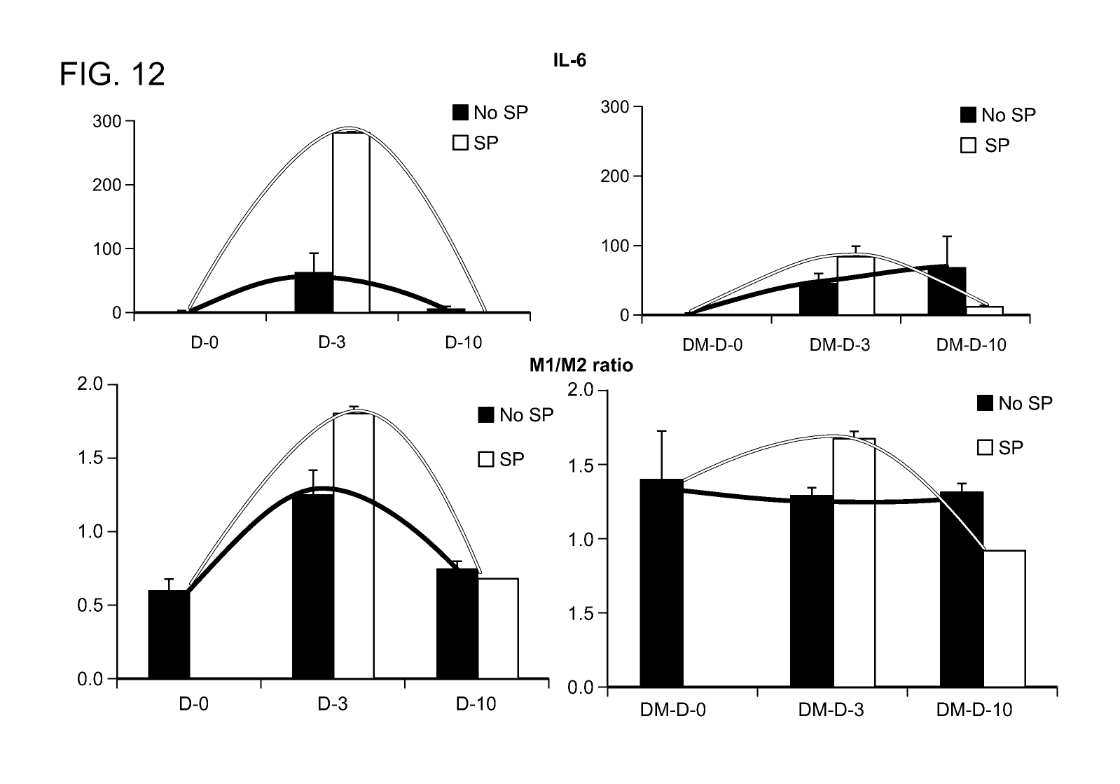

Daily topical Substance P (SP) application in 6 mm excisional wounds created at the back of non-DM and STZ-DM mice increased the IL-6 expression and M1/M2 ratio three days after wounding, imitating the acute inflammatory phase of wound healing in acute wounds (FIG. 12). However, at Day-10, SP reduced both these inflammatory factors in STZ-DM mice, which was increased when compared to non-DM mice. These results support that local SP treatment restores the wound healing dynamics and, even perhaps more importantly, reduces the long-term diabetes-related chronic skin inflammation.

IENFD is Reduced WT STZ-DM and TAC1KO Mice and Topical SP or DSCG Treatment in STZ-DM Mice Restores it.

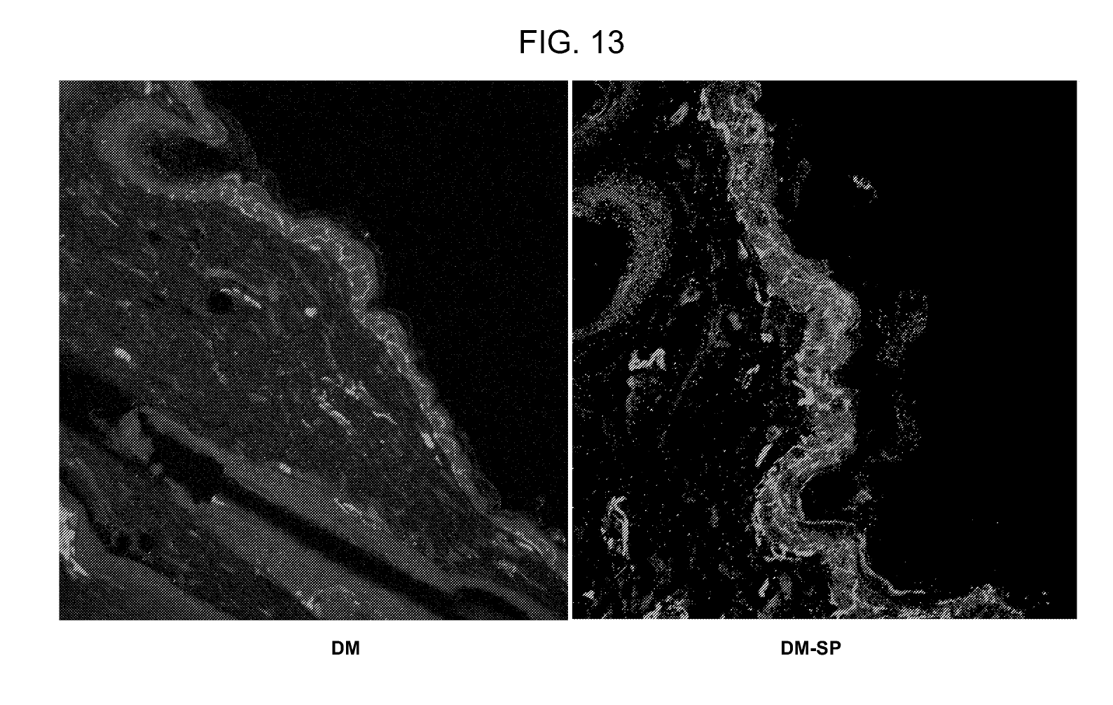

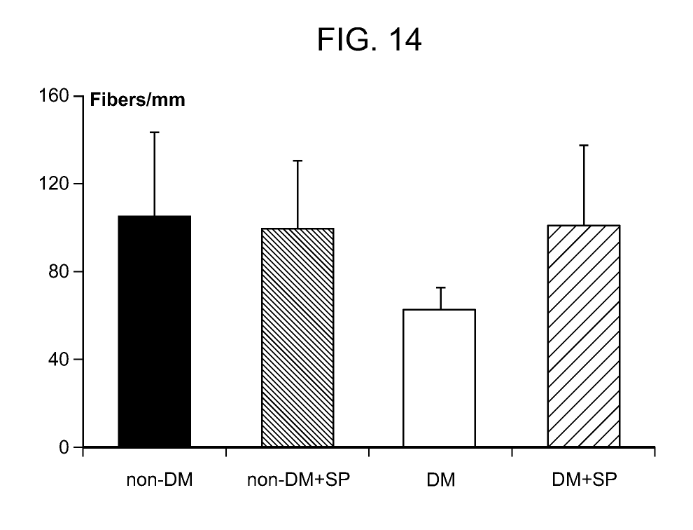

In a blinded mode, the IENFD in normal skin areas adjacent to wounds in non-DM and DM not-treated and treated with topical SP application for a 10-day period as described above (FIGS. 13 and 14) was evaluated. SP treatment in non-DM mice had no obvious effect on IENFD. However, DM mice trended to have lower IENFD and SP treatment increased IENFD to levels similar to the non-DM mice. Similar results were found in preliminary studies that evaluated the effect of MC stabilization with DSCG. More specifically, DSCG treatment in two DM mice tended to have a beneficial effect in IENFD (151±55 fibers/mm) compared to four non-treated DM mice (55±17, p<0.05). Initial observations in TAC1KO mice also indicated reduced IENFD (28±12, n=2 mice) that improved in a similar way as with the previous experiments following SP treatment (41±16, n=2).

### III. Efficacy of Topical SP Treatment in Preventing and Reversing SFN in Type 1 DM and Type 2 DM Animal Models.

Based on the previously demonstrated biocompatibility of alginate hydrogels and generic DNA nanomaterials (see e.g., Proc Natl Acad Sci USA 2012; 109:19590-5, Biomaterials 2010; 31:1235-41, and Advanced materials 2011; 23:1117-21), alginate hydrogels embedded with DNA nanocontainers for controlled topical skin SP release is contemplated herein. One object is to decorate with SP a DNA nanostructure that is designed, based on size and shape, to delay internalization of bound NK1 receptors, thereby prolonging their activation. In addition, as elongated structures can give rise to greater NK1R activation, another objective is to decorate different shape DNA nanostructures with the same amounts of SP and investigate the NK1R activation. Additional objects include e.g., development of a more sophisticated nanostructure that only displays SP after allosteric activation of a second domain, something that will allow specific targeting of the intraepidermal nerve fibers.

Efficacy of Topical and Systemic MC Stabilization Treatment in Preventing SFN in T1DM and T2DM Animal Models.

As shown by the data above, both human and experimental diabetes are associated with increased MC degranulation. Recent work has also shown that MC deficiency is associated with impaired wound healing and abrogates the beneficial effects of SP in wound healing. See e.g., Wound Repair and Regeneration 2013; 21:A45-A and Diabetologia 2011; 54:S471-S. Treatment with DSCG reverses these abnormalities and also reduces the M1/M2 ratio to normal levels. These results are compatible with other studies that have shown that MC stabilization reduces obesity, inflammation and the macrophage infiltration of the adipose tissue in mice fed with western diet. See e.g., Nat Med 2009; 15:940-5. Mast cell stabilization, either local or systemic, will reduce skin inflammation should therefore prevent the development of SFN.

MC degranulation is controlled by elevated levels of cytosolic calcium that is mediated by the stored operated calcium (SOC), and to a lesser extent, by the receptor potential canonical (TRPC) channels. The best characterized SOC channel is the calcium selective orai, also known as calcium-release activated calcium (CRAC) channel that is expressed by MC. Activation of MC stimulates the opening of the orai channels for calcium influx. The critical role of orai/CRAC channel in MC effector function is substantiated by the fact that their genetic ablation severely impaired MC degranulation and the release of pro-inflammatory mediators. See e.g., Nature immunology 2008; 9:89-96. Small molecule orai/CRAC channel blockers are shown to potently inhibit MC degranulation (The international journal of biochemistry & cell biology 2011; 43:1228-39) and T-cell activation (The Journal of biological chemistry 2001; 276:48118-26). Selective orai channel blockers are described in e.g., (WO2010/039036 and WO2005/009954. Dual orai/TRPC channel blockers are also contemplated herein.

Efficacy of Topical Combined SP and MC Stabilization Treatment in Preventing and Reversing SFN in Type 1 DM and Type 2 DM Animal Models.

Ongoing studies have shown that TAC1KO mice have increased MC degranulation when compared to WT mice, while the induction of DM does not induce an increase in the MC degranulation similar to the one that induces in WT mice. In addition, topical wound SP treatment in MC deficient mice lacks the beneficial effects that are seen in the wound healing of WT mice. These results suggest the already known interaction between SP and MC, something that can also be expected by the fact that MC express the NK1R receptor (J Invest Dermatol 2007; 127:362-7, Wound Repair Regen 1998; 6:8-20, and Proc Natl Acad Sci USA 2010; 107:4448-53). Combined topical treatment with SP and an MC stabilization agent should have an additive, if not synergistic, effect in preventing and reversing SFN.

While we have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of delaying the onset of, reversing, or reducing the risk of acquiring peripheral neuropathy (PN) in a subject having diabetes, comprising administering to the subject a therapeutically effective amount of a Substance P, a mast cell (MC) degranulation inhibitor, or a combination thereof.

2. The method of claim 1, wherein the peripheral neuropathy is small fiber neuropathy (SFN).

3. The method of claim 1, wherein the subject has diabetes mellitus type 2.

4. The method of claim 1, wherein the Substance P, mast cell degranulation inhibitor, or combination thereof is administered topically.

5. The method of claim 1, wherein the mast cell degranulation inhibitor is a calcium channel blocker or receptor potential canonical (TRPC) channel blocker.

6. The method of claim 1, wherein the mast cell degranulation inhibitor is a calcium-release activated calcium (CRAC) channel blocker.

7. A method of delaying the onset of, reversing, or reducing the risk of acquiring peripheral diabetic neuropathy in a subject in need thereof, comprising administering to the subject a therapeutically effective amount of a Substance P, a mast cell (MC) degranulation inhibitor, or a combination thereof.

8. The method of claim 7, wherein the peripheral diabetic neuropathy is caused by diabetes mellitus type 2.

9. The method of claim 7, wherein the mast cell degranulation inhibitor is administered topically.

10. The method of claim 7, wherein the mast cell degranulation inhibitor is a calcium channel blocker or receptor potential canonical (TRPC) channel blocker.

11. The method of claim 9, wherein the mast cell degranulation inhibitor is a calcium-release activated calcium (CRAC) channel blocker.

12. A method of delaying the onset of, reducing the risk of developing, or accelerating the healing of a wound in subjects having diabetes, comprising administering to the subject a therapeutically effective amount of Substance P, a mast cell (MC) degranulation inhibitor, or a combination thereof.

13. The method of claim 12, wherein the wound is a foot ulcer.

14. The method of claim 12, wherein the subject has diabetes mellitus type 2.

15. The method of claim 12, wherein the Substance P, mast cell degranulation inhibitor, or combination thereof is administered topically.

16. The method of claim 12, wherein the mast cell degranulation inhibitor is a calcium channel blocker or receptor potential canonical (TRPC) channel blocker.

17. The method of claim 12, wherein the mast cell degranulation inhibitor is a calcium-release activated calcium (CRAC) channel blocker.

18. A method of altering the M1/M2 macrophage ratio in a wound on a subject having diabetes, comprising administering to the subject a therapeutically effective amount of Substance P, a mast cell (MC) degranulation inhibitor, or a combination thereof.

19. The method of claim 18, wherein the M2 macrophage becomes polarized.

20. The method of claim 18, wherein the M1/M2 macrophage ratio is reduced.

21. The method of claim 18, wherein the wound is a foot ulcer.

22. The method of claim 18, wherein the subject has diabetes mellitus type 2.

23. The method of claim 18, wherein the Substance P, mast cell degranulation inhibitor, or combination thereof is administered topically.

24. The method of claim 18, wherein the mast cell degranulation inhibitor is a calcium channel blocker or receptor potential canonical (TRPC) channel blocker.

25. The method of claim 18, wherein the mast cell degranulation inhibitor is a calcium-release activated calcium (CRAC) channel blocker.

26. A method of preventing the increase of matrix metallopeptidase 9 (MMP-9), in subject having diabetes, comprising administering to the subject a therapeutically effective amount of Substance P, a mast cell (MC) degranulation inhibitor, or a combination thereof.

27. The method of claim 26, wherein preventing the increase of matrix metallopeptidase 9 accelerates the healing of a wound.

28. The method of claim 26, wherein preventing the increase of matrix metallopeptidase 9 accelerates the healing of a foot ulcer.

29. The method of claim 26, wherein the subject has diabetes mellitus type 2.

30. The method of claim 26, wherein the Substance P, mast cell degranulation inhibitor, or combination thereof is administered topically.

31. The method of claim 26, wherein the mast cell degranulation inhibitor is a calcium channel blocker or receptor potential canonical (TRPC) channel blocker.

32. The method of claim 26, wherein the mast cell degranulation inhibitor is a calcium-release activated calcium (CRAC) channel blocker.
